(19) United States
(12) Patent Application Publication
(10) Pub. No.: US 2006/0037617 A1
(43) Pub. Date: Feb. 23, 2006

(54) Airway implant devices and methods of use

(76) Inventors: Amrish Jayprakash Walke, Menlo Park, CA (US); Jeremy Johnson, San Francisco, CA (US); Russell Woo, Palo Alto, CA (US); Evan Anderson, San Francisco, CA (US); Kelly Richardson, Menlo Park, CA (US); Kenneth Martin, Los Gatos, CA (US)

Correspondence Address: BELL & ASSOCIATES, 416 FUNSTON ST., SUITE 100, SAN FRANCISCO, CA, 94118, US

(21) Appl. No.: 11/110,104

(22) Filed: Apr. 19, 2005

Related U.S. Application Data
(60) Provisional application No. 60/563,604, filed on Apr. 19, 2004.
(60) Provisional application No. 60/620,076, filed on Oct. 20, 2004.

(51) Int. Cl. A62B 9/06; A61M 16/00

(52) U.S. Cl. 128/207.15; 128/207.14

(57) ABSTRACT

The invention is drawn to airway implant devices that can alleviate and remediate the effects of dysphagia and aspiration that occur under a wide range of clinical conditions, such as Parkinson's disorder, Alzheimer's disease, or stroke. The devices can further be used to reduce the risk of onset of aspiration pneumonia in these and many other clinical conditions.

The present application claims priority to U.S. Provisional Patent Application Ser. No. 60/563,604 entitled “Dysphagia Interventions”, filed Apr. 19, 2004, and U.S. Provisional Patent Application Ser. No. 60/620,076 entitled “Tracheal Devices and Methods”, filed Oct. 20, 2004, which are herein incorporated by reference in their entirety for all purposes.

### FIELD OF THE INVENTION

The present invention relates to devices and methods to prevent or treat or reduce the risk in an individual of aspiration pneumonia and related conditions.

### BACKGROUND

Dysphagia is a condition in which a subject is not able to properly swallow. Solid and liquid food as well as oral secretions are not properly diverted into the esophagus, and enter the lungs through the trachea. This ultimately causes infection in the lungs that leads to a serious and life-threatening condition called aspiration pneumonia. Aspiration pneumonia is a very common disease, particularly among people suffering from stroke, Parkinson's disorder (PD), and Alzheimer's disease (AD).

During normal swallowing, food and liquids enter the stomach via the esophagus and are prevented from entering the airway (trachea, bronchi, and lungs). In individuals or subjects having dysphagia, this normal process is disrupted and results in leakage of food particles, oral secretions, and/or stomach contents into the lungs. This later process, termed aspiration, can further result in a life-threatening inflammation and/or infection of the lungs termed aspiration pneumonia.

Although dysphagia and aspiration pneumonia represent significant clinical, social, and economic costs and issues, no effective therapies currently exist to prevent and/or treat these urgent medical needs. In the United States alone, approximately 800,000 individuals per year are affected by dysphagia that is a consequence of neurological disorders (for example, stroke, PD, and AD). Stroke survivors alone can account for about 100,000 cases of aspiration. Studies have suggested that the rate of dysphagia in PD patients is approximately 69%. Other disorders also induce a high incidence of dysphagia, such as AD (84%), motor neuron disease (51%), amyotrophic lateral sclerosis (ALS) (29%), progressive supranuclear palsy (56%), and Huntington's disease (HD) (100%). In addition aspiration pneumonia may also be caused during periods of altered consciousness, often when a person is affected by drugs or alcohol, or after head injury or anesthesia.

The oral-tracheal pathway can be schematically envisioned as including a junction having three potential fluid pathways: the oropharyngeal pathway, the tracheal pathway, and the esophageal pathway. Solids and liquids (food, drink, saliva, and mucus) from the oral cavity and nasopharynx enter the oropharyngeal pathway. In dysphagia, the solids and fluids then inappropriately enter the trachea instead of the esophagus.

There is therefore a need in the art for use in the biomedical, clinical, and surgical arts to provide devices and methods that can prevent aspiration in individuals having dysphagia.

### BRIEF DESCRIPTION OF THE INVENTION

The present invention provides an airway implant device for collecting aspirate, the device comprising a collector, the collector comprising at least one lumen and at least one drain, the device being adapted for placement within the trachea of an individual having aspiration. In one embodiment, the airway implant device further comprises a channel. In an additional embodiment, the airway implant device further comprises at least one stiffening element, wherein the element comprises a material selected from the group consisting of coated TEFLON, high-density polyethylene (HDPE), low-density polyethylene (LDPE), polyamide, nylon, polytetrafluoroethylene (PTFE), polyetheretherketone (PEEK), or the like. In another embodiment, the airway implant device comprises a material, the material selected from the group consisting of NITINOL, stainless, steel, and titanium. In a yet further embodiment, the collector comprises an elastomeric material, the elastomeric material selected from the group consisting of polyurethane, poly(vinyl)chloride (PVC), silicone rubber, latex, or the like. In another embodiment of the invention, the collector comprises a swellable polymeric material, the swellable polymeric material comprising a swelling gel that swells and expands in volume when it comes into fluid contact with water. Preferably the swelling gel is a super absorbent polymer. Most preferably the super absorbent polymer is sodium polyacrylate.

In another embodiment the airway implant device comprises a collector further comprising securing means, the securing means adapted for securing the collector to the individual's tissue, thereby securing the airway implant device within the trachea. In one embodiment, the securing means is selected from the group consisting of sutures, clips, hook, staples, stents, adhesives, and the like, a chemical foam, an inflatable balloon, an inflatable cuff, a flange, existing intra-tracheal securing devices known in the art, and the like. In a more preferred embodiment, the chemical foam is selected from the group consisting of hydrophilic foams, hydrophobic foams, and biodegradable foams. In a most preferred embodiment, the chemical foam is selected from the group consisting of polyurethane, polyhydroxyalkanoate (PhA), polyhydroxybutyrate (PhB), PVC, and PTFE.

In yet another embodiment of the invention, the airway implant device further comprises delivering means, the delivering means having deploying means, the deploying means having releasable attaching means securing the collector. In a preferred embodiment, the delivering means is selected from the group consisting of a laryngoscope and a bronchosope. In an alternative embodiment, the delivering means comprises a puncturing means, the puncturing means shaped and adapted for puncturing the skin, fascia, cartilage, and tracheal tissue of the individual. In a further embodiment of the invention the delivering means further comprises a loop. In a still further embodiment, the invention provides a hook that is shaped and sized to be suitable for placement in the airway of an individual and that is then used to secure the loop.

In another embodiment of the invention, the airway implant device comprises a collector said collector further comprising a first lumen and a second lumen and wherein the collector comprises an elastomeric material.

An alternative embodiment of the invention provides an airway implant device comprising a first arm, a second arm, a third arm, and a baffle, the baffle shaped and adapted for placement in the laryngopharynx of an individual having tracheal aspirate, and wherein the baffle is further shaped and adapted for diverting tracheal aspirate from flowing into the trachea.

In use the airway implant device of the invention provides an individual having aspiration an improved ability to breathe and to talk.

The invention also provides a method of using an airway implant device to treat an individual having aspiration, the method comprising the steps of: (i) providing an individual having aspiration, (ii) providing an airway implant device, (iii) attaching the airway implant device to attaching means, (iv) further attaching the attaching means to deploying means, (v) implanting the airway implant device in the trachea of the individual using deploying means, (vi) releasing the airway implant device from the attaching means, and (vii) securing the airway implant device to the wall of the trachea using securing means, thereby treating the individual having aspiration. The method also results in the patient having an improved ability to talk and to breathe.

### BRIEF DESCRIPTION OF THE DRAWINGS

FIG. 1 shows an illustration of one embodiment of the invention. FIG. 1A shows the collector, the channel, the lumen, and the drain. FIG. 1B shows the stiffening rod positioned within the collector material. FIG. 1C shows the device in place showing how liquid flows down the tracheal wall, collects in the channel, and is drained from the collector.

FIG. 2 shows an alternative embodiment of the airway implant device.

FIG. 3 shows a cross-sectional diagram showing different embodiments of securing means that secure the device to the inner wall of the trachea.

FIG. 4 shows a three-quarter view of the device positioned in a stent.

FIG. 5 shows a cross-sectional view of a device the invention being deployed using a laryngoscope.

FIG. 6 shows a cross sectional view of a device of the invention being deployed using a bronchoscope.

FIG. 7 shows another embodiment of the airway implant device. FIG. 7A shows elements of the device. FIG. 7B shows the device in use in the laryngopharynx, showing how aspirate is deflected away from entering the trachea.

FIG. 8 shows a cross section of another embodiment of the airway implant device being deployed in use.

FIG. 9 shows a cross section of another embodiment of the invention being deployed in use.

FIG. 10A shows a cross sectional illustration of an airway device positioned in the trachea in a deflated or unexpanded state. FIG. 10B shows a cross sectional illustration of an airway device in an inflated or expanded state.

FIG. 11 shows three embodiments of the airway implant device, showing different arrangements for inflating or expanding the device in the trachea.

FIG. 12 shows a cross sectional illustration showing the airway implant device of the invention in an un-deployed state (12A) and in a deployed state (12B) when positioned in use in the trachea.

FIG. 13 shows an embodiment of the airway implant device of the invention that can accommodate placement within the vocal chords of an individual.

FIG. 14 shows an alternative embodiment of an airway implant device for use positioned within the vocal chords.

FIG. 15 shows the device of FIG. 14 positioned within open (15A) vocal folds or closed (15B) vocal folds.

FIG. 16 shows an illustration of an alternative embodiment of the airway implant device of the invention in use illustrating how pressurized air is forced through apertures adjacent to the channel for the purpose of redirecting any aspirate that may fall through the center of the device into the collector.

FIG. 17 shows an illustration of an alternative embodiment of the airway implant device. FIG. 17A shows a three-quarters view from one aspect. FIG. 17B shows a three-quarters view from another aspect. FIG. 17C shows the device positioned in the oropharynx and laryngopharynx of an individual and showing differential segregation of solid/liquid and gas pathways flowing either to the esophagus or to the trachea, respectively.

FIG. 18 shows an illustration of the experimental equipment setup to test an embodiment of the airway implant device.

FIG. 19 shows an embodiment of the airway implant device showing dimensions of the device that was used in the experimental setup.

FIG. 20 shows the results of the experiment that tested the airway implant device.

### DETAILED DESCRIPTION OF THE INVENTION

The embodiments disclosed in this document are illustrative and exemplary and are not meant to limit the invention. Other embodiments can be utilized and structural changes can be made without departing from the scope of the claims of the present invention.

As used herein and in the appended claims, the singular forms “a,” “an,” and “the” include plural reference unless the context clearly dictates otherwise. Thus, for example, a reference to “a channel” includes a plurality of such channels, and a reference to “a baffle” is a reference to one or more baffles and equivalents thereof, and so forth.

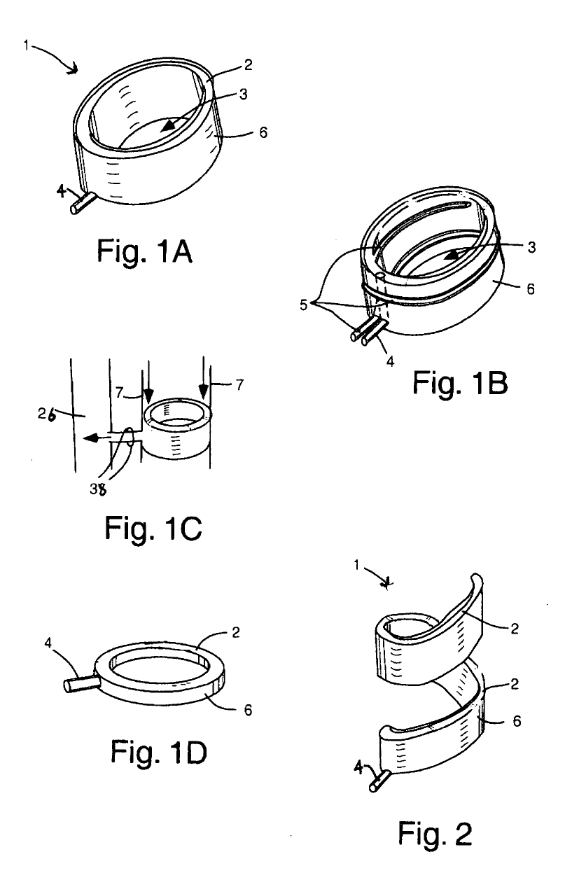

In one embodiment, the airway implant device 1 comprises a collector 6 for collecting aspirate and a drain 4 for ducting aspirate away from the device and away from the tracheal lumen (see FIG. 1D and FIG. 2).

Alternatively, the airway implant device 1 comprises a channel 2 for collecting aspirate, a lumen 3 through which air or other gases can flow, a drain 4 for ducting aspirate away from the device and the tracheal lumen (see FIG. 1A, and FIG. 1C).

In another alternative, the airway implant device 1 further comprises a stiffening means 5. The stiffening means is integrated within the material comprising the collector and results in a collector having improved structural properties. The structural properties include, but are not limited to, resilience to torque, resilience to movement within the lumen of the trachea, resilience to deformation, and the like. The stiffening means comprises a resilient material, such as, but not limited to, metal such as stainless steel, NITINOL, titanium, or the like, a material that is substantially plastic, such as DELRIN (acetal), acrylonitrile butadiene styrene (ABS), nylon, polypropylene, polycarbonate, glycolised polyethylene terephthalate (PETg) copolyester, polyvinyl chloride (PVC), or the like, and can be easily machined or injection molded to the required shape (see FIG. 1B).

In use, the airway implant device is positioned within the airway of an individual having aspiration. In an individual having aspiration, the aspirate flows along the inner wall of the trachea 7 and into the collector or the channel. The aspirate is then conducted to the drain. (See FIG. 1C; direction of flow—solid arrowhead.)

The aspirate collected in the device can be removed from the drain using additional conducting means, such as a substantially flexible tube having a lumen. The substantially flexible tube is surgically positioned so that the distal open end is releasably secured to the drain outlet. The tube is then surgically positioned to traverse at least one tissue layer, for example, the tracheal wall, the esophageal wall, muscle, cartilage, fascia, and/or the skin, and the like. The proximal open end of the tube is placed in fluid communication with a container, such as a sump, a trap, a reservoir, or the like. The aspirate then flows under the force of gravity from the collector to be stored in the container. In use, the aspirate is only temporarily stored in the container prior to disposal.

In one other alternative use, the medial portion of the tube traverses the wall of the esophagus via a fistula 38 and the proximal end of the tube is surgically positioned in the lumen of the esophagus (see FIG. 1C). The aspirate can then flow under the force of gravity to the esophagus 26.

In addition, in an alternative use, the drain is so positioned with regard to the collector to ensure that the collected aspirate is channeled or ducted away from the device and/or the trachea by securing the distal end of a tube having a lumen having a generally similar diameter to the diameter of the drain outlet to said outlet. The proximal end of the tube is in fluid communication with a device, such as a vacuum pump, that evacuates gases or air to create a local vacuum. Under the local negative pressure induced by the vacuum device, the aspirate flows through the collector, the drain, and the tube towards the vacuum device. The vacuum device can further comprise a sump, trap, or the like, where the aspirate is stored pending later disposal. In an additional alternative, the airway device can comprise a drain in fluid communication with such a vacuum device via a tube and the vacuum being of sufficient negative pressure to evacuate aspirate from the trachea in the absence of collecting or channeling means.

The aspirate can be drained and conducted away from the trachea through a number of different anatomical sites, for example, the anterior region of the trachea and the anterior neck region, through a tracheal-esophageal fistula, through and/or into the stomach, through and/or into the upper gastrointestinal tract, into and through the mouth (oral cavity), and into and through the nose (nasal cavity).

In an alternative embodiment, the collector can comprise a single wall, a lumen, a drain, and a flange, the flange positioned at the base of the collector thereby providing both means to secure and/or attach to the wall of the trachea and means to collect aspirate that flows across the surface of the wall of the trachea. The flange conducts the aspirate to the drain.

The airway implant device can also comprise a sensor, the sensor having means to detect the presence of aspirate. Such means are well known to those of skill in the art and can include, but are not limited to, pressure sensors, touch sensors, biological sensors (for example a sensor that detects mucus fluids), chemical sensors (for example a sensor that detects an electrolyte or the like), optical sensors (for example a sensor that detects a change in refractance of incident light), sensors that are activated by an electric circuit closed by an aqueous solution, or the like.

The airway implant device can be implanted, placed, and/or positioned in the body for a temporary period of between minutes to years or can be permanently implanted.

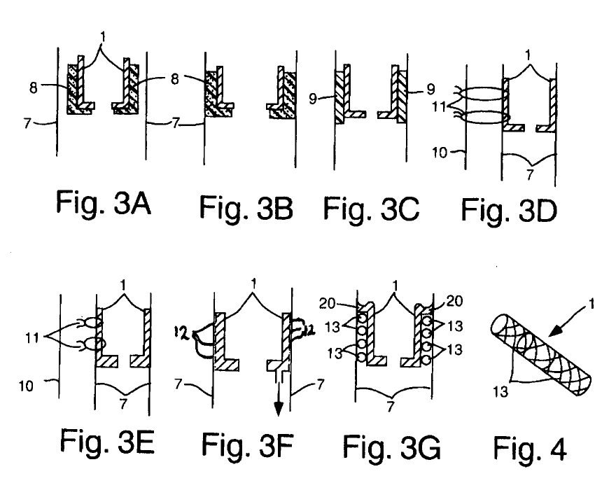

The airway implant device can be sealed, secured and/or attached to the wall of the trachea 7 or airway using securing or attaching means well known to those in the art. Such means can include, but are not limited to, sutures 11, clips, hook, staples, stents 13, adhesives 9 and the like, a swellable gel, a chemical foam 8, inflatable means 8 such as an inflatable balloon or an inflatable cuff, a flange 20, existing intra-tracheal securing devices known in the art, and the like. (See FIG. 3 and FIG. 4.)

FIG. 3A shows a cross-sectional illustration of an airway implant device 1 further comprising inflatable means, the inflatable means being uninflated. As shown in FIG. 3A, the airway implant device is not in contact with the wall of the trachea 7. FIG. 3B shows the inflatable means inflated and the device of the invention is secured against the wall of the trachea.

FIG. 3C shows the airway implant device further comprising adhesive means 9. Adhesive means for surgical use are well known to those of skill in the art, and are for example biocompatible adhesives including, but not limited to, cyanoacrylate adhesives, fibrin sealants, chemically-modified natural proteins such as, but not limited to, collagen or albumin further comprising aldehyde cross-linking agents such as, but nor limited to, glutaraldehyde or formaldehyde, gelating-resorcinol-formol glues, and the like.

Advantages of using a biocompatible glue or a foam or any adhesive, for example are that there is expected to be no necrosis of the tracheal wall tissue. Necrosis is caused by pressure cutting off the blood supply of the capillary beds under the contact point. Another advantage is that there can be little or no migration of the device medially or laterally with respect to the wall of the trachea. A considerable pulling force would be required or expected expected to dislodge the device so positioned. A further advantage of using a biocompatible glue or foam or any adhesive can be that the tracheal wall dynamics are unaltered. The trachea can move naturally since there is no pressure against it and it would not interfere with speech or with normal swallowing.

The biocompatible glue may be deployed, for example, by a device that expands the stiffening means thereby pushing the outward wall of the device against the trachea for long enough for glue to set. Alternatively, the glue may be delivered by, for example, a porous material that is part of the outer edge of the device and the glue may be pumped out from within the device. In another alternative, a breakable capsule containing the glue is in the device and the capsule is broken remotely by an operator. Glue can also be delivered via tubing from outside the individual having aspiration. The device may comprise an adhesive outer surface, for example, a sticker comprising an adhesive and a protective material whereby the protective material is peeled away to reveal the adhesive surface

Securing means can also comprise a swellable gel, wherein the collector comprises a material that swells when it comes into contact with water. The material can be made of crosslinked polymers. Examples of such polymers that can be used include. but are not limited to, sodium acrylate polymer, crosslinked sodium polyacrylate polymer, acrylamide polymer, acrylamide derivative polymer or copolymer, sodium acrylate and vinyl alcohol copolymer, crosslinked polyethylene oxide, isobutylene-maleic anhydride crosslinked copolymer, vinyl acetate and acrylic acid ester copolymer, vinyl acetate and methyl maleate copolymer, starch-acrylonitrile graft copolymer, or mixtures thereof, and any other similar crosslinked polymers or copolymers.

FIGS. 3D and 3E show cross-sectional illustrations of an airway implant device wherein the securing or attaching means are surgical sutures 11. FIG. 3D shows the sutures securing the device wherein the suture is surgically passed through the tracheal wall 7 and the skin 10 of the individual and secured thereto. FIG. 3E shows an alternative surgical procedure wherein the suture is passed though the tracheal wall 7 and secured thereto. Suitable surgical sutures that can secure an implant device are well known to those in the art and can include, cotton, animal mesentery, animal collagen, other animal fibers, plant fibers, and the like.

FIG. 3F shows another alternative airway implant device wherein the outer wall of the collector further comprises a plurality of apertures 12 and a gas outlet. The apertures are distributed in the outer wall in such a manner so as to provide movement or a gas or air therethrough. In use, the gas outlet is releasably secured in fluid communication with the distal end of a flexible tube having a lumen, the proximal end of the flexible tube releasably secured to a vacuum pump. The flexible tube comprises a flexible material such as, but not limited to, latex, polyethylene, polyurethane, nylon, TYGON (US Plastic Corp., Lima Ohio), and CHEMFLUOR (US Plastic Corp.). In operation the vacuum pump is switched on and a negative pressure gradient is created, the air in the collector is drawn to the vacuum pump (see FIG. 3F, arrow), air from within the trachea is drawn through the apertures and the collector wall of the implant device is displaced towards the tracheal wall and remains in position due to the lower gas or air pressure between the surface of the tracheal wall and the surface of the outer wall of the collector.

In yet another alternative, shown in FIG. 3G and FIG. 4, the airway implant device 1 further comprises a stent 13, whereby the stent is shaped and adapted to conform to the diameter of the trachea wall 7 when expanded.

A further alternative airway implant device comprises a flange 20, wherein the flange is shaped and adapted for placement against the tracheal wall thereby providing securing means against the tracheal wall and securing the device in place (see FIG. 3G). The flange 20 can be used with any of the alternative embodiments of the device of the invention disclosed herein.

The body of the device 1 may have a diameter of, for example, from 3 mm to 40 mm, or from 3 mm to 35 mm, or for example about 3 mm, 7 mm, 12 mm, 25 mm, 18 mm, 22 mm, 25 mm, 28 mm, 31 mm, 35 mm, or 40 mm. The length of the device may be any length compatible with its function of maintaining a clear airway pathway, and the device may (or may not) be shorter than the deploying device and/or system that is used to deploy it into the tairway. For example, the device may be from 4 cm to 30 cm in length, or for example about 5 cm, 7 cm, 10 cm, 14 cm or 18 cm in length. The body of the invention may be of variable fixed lengths, or it may be of dynamically adjustable length by use of a telescoping designs. The body of the invention is generally a flat or elongated cylinder, though it may be of any suitable cross-sectional shape such as oval or polygonal. The body of the invention may be rigid or may be flexible. A flexible body is desirable when using a flexible endoscope.

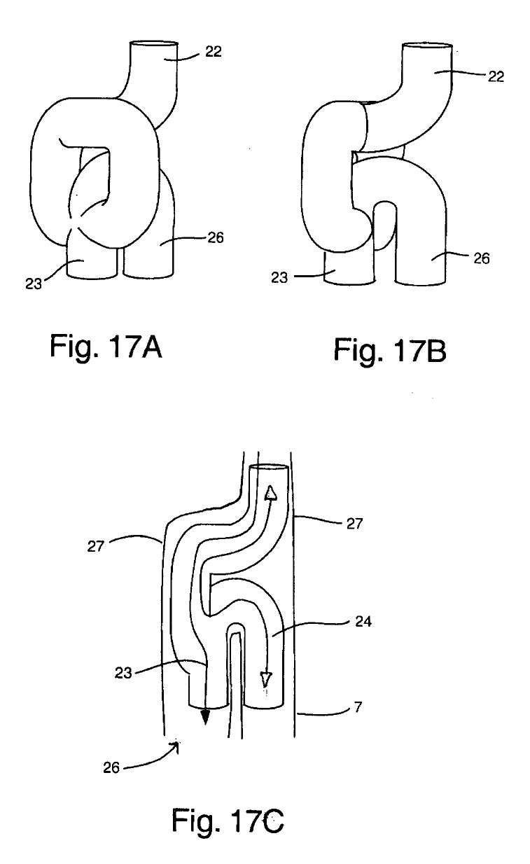

The airway implant device of the invention can also alternatively comprise a device that is shaped and adapted for positioning within the laryngopharynx, the larynx, and the esophagus. Examples of such devices are shown in FIG. 7 and FIG. 17. As shown in FIG. 7A, the device comprises a first arm 22, a second arm 23, a third arm 24, and a baffle 25. In use, the device is positioned in the laryngopharyngeal cavity using any deploying means described herein as shown in FIG. 7B. The first arm 22 is positioned below the oropharnyx and above the laryngopharyn 27 and the esophagus 26 and the trachea. The second arm 23 is positioned in the esophagus and the third arm 24 is positioned in the trachea. In use, the baffle 25 directs solid matter, such as food, aspirate, or water (solid arrowhead), to the esophagus 26; gases, such as air or gases used in anesthetic methods (open arrowheads), are free to pass through the lumen of the device around the baffle to the trachea (tracheal wall 7 shown).

As shown in FIG. 17A and FIG. 17B, the device comprises a first arm 22, a second arm 23, a third arm 24. In this embodiment, the first arm 22 splits into two lumens. In use, the device is positioned in the laryngopharyngeal cavity using any deploying means described herein as shown in FIG. 17C. The first arm 22 is positioned below the oropharnyx and above the laryngopharynx 27 and the esophagus 26 and the trachea. The second arm 23 is positioned in the esophagus and the third arm 24 is positioned in the trachea. In use, solid matter, such as food, aspirate, or water (solid arrowhead), are directed by the shape of the first arm lumens to the esophagus 26; gases, such as air or gases used in anesthetic methods (open arrowheads), are free to pass through the lumen of the device to the trachea (tracheal wall 7 shown).

The alternative devices disclosed in FIG. 7 and FIG. 17 can also comprise a drain in fluid communication with the second arm of the device, the drain that can be routed through an incision in the thorax or the neck of an individual having aspiration and thereby conduct the aspirate away from the body.

FIG. 16 shows an alternative embodiment of the airway implant device, wherein the device further comprises a reservoir 36, the reservoir further comprising a plurality of apertures 35 and at least one inlet 38. In use, the inlet is placed in fluid communication with a pressure device, such as an air pump, whereby air is pumped under pressure through the inlet into the reservoir (solid arrowhead). The air then escapes from the reservoir via the apertures in a plurality of air streams (solid arrowheads). Using a pressure predetermined by the operator, the escaping air flows close to the surface of the trachea, thereby forcing the aspirate away from the lumen and into the collector or channel. The aspirate is conducted from the collector or channel through the drain as described herein (open arrowhead).

The airway implant device can be manufactured so as to conform to an individual's own trachea, i.e. can be custom-molded. The shape and size of the device is determined using measurements taken from, for example, an electromagnetic scan of the patient's anatomy using imaging technology such as MRI, CAT scans, or the like.

Deploying the Airway Implant Device

The airway implant device if the invention can be deployed by an operator using any of the deploying means in use. Such deploying means include, but are not limited to, a laryngoscope, a bronchoscope, a laparoscope, a catheter, a stent, a needle, or the like. In addition, the airway device can be deployed by surgical implantation through a tracheotomy, through the oral cavity, or from other locations within the thoracic cavity and thoracic wall. The airway device may also be implanted using the natural swallowing reflex of the individual having aspiration.

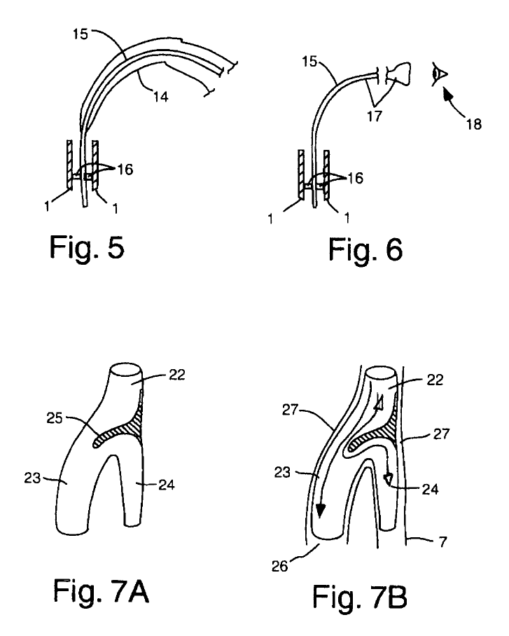

FIG. 5 and FIG. 6 show two exemplary embodiments of how the device can be deployed by an operator. FIG. 5 shows a laryngoscope 14 that can be inserted into the oral cavity of an individual to assist an operator deploy the airway implant device 1. FIG. 6 shows a bronchoscope that can be inserted into the oropharyngeal cavity of an individual to assist an operator deploy the airway implant device 1.

The deployment device can also comprise a deploying means 15 and releasable securing or attaching means 16. The airway implant device is releasable secured to the deploying means 15 using the releasable securing or attaching means 16. Deploying means can be an endotracheal tube, a catheter, a tube, a cable, a guidewire, or the like. Releasable securing or attaching means can be a clip, a pin, a fastener, a clasp, a tie, or the like.

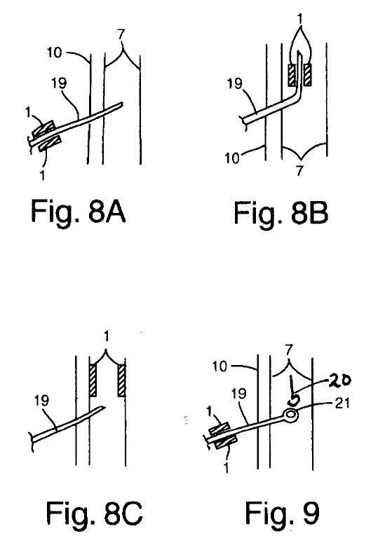

The device can also be deployed by surgical insertion and implantation of the device through the skin of the individual. FIG. 8 shows an illustration of an exemplary procedure. A catheter 19 is inserted by an operator through an incision in the skin and tracheal wall of the individual having aspiration (see FIG. 8A). The distal end of the catheter can further comprise a marker, such as a light source and/or a radio-opaque material, to assist the operator to position the airway implant device in the desired position in the airway. The airway implant device 1 is moveably positioned on the proximal end of the catheter using the catheter as a guidewire (FIG. 8A) and advanced through the skin and tracheal incision to the distal end of the deployment means (FIG. 8B). The device can additionally be attached to the catheter using any of the deploying means described herein. The device is deployed and positioned in the trachea and the released by the operator and the catheter withdrawn from the device (FIG. 8C).

FIG. 9 shows yet another exemplary deployment means that can be inserted by an operator through an incision in the skin and tracheal wall of the individual having aspiration. The deploying means comprises a loop 21. The distal end of the loop can further comprise a marker, such as a light source and/or a radio-opaque material, to assist the operator to position the airway implant device in the desired position in the airway. The operator inserts a hook 20 via the oropharyngeal cavity of the individual, and grasps the loop using the hook. The device 1 is releasably attached to the catheter 19 and can be drawn through the incision by the operator and positioned in the trachea. The airway implant device is deployed as described herein. The loop catheter and the hook can be unhooked and retracted by the operator. In an alternative embodiment the hook is positioned on the catheter and the loop is inserted through the oropharyngeal cavity by the operator

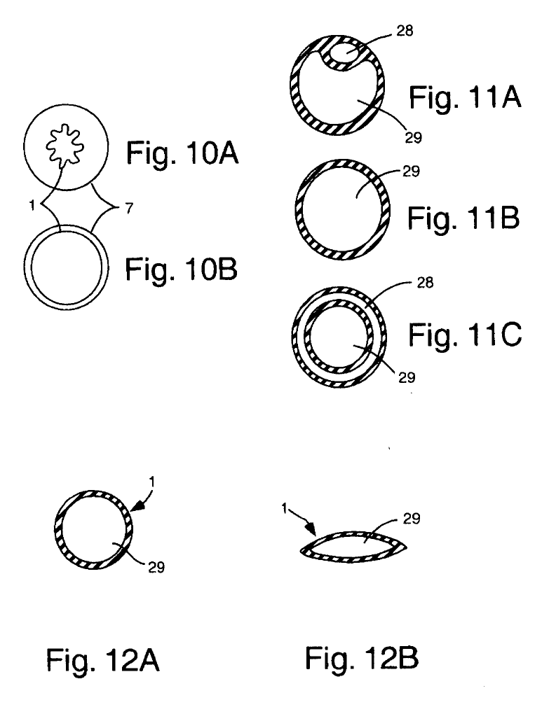

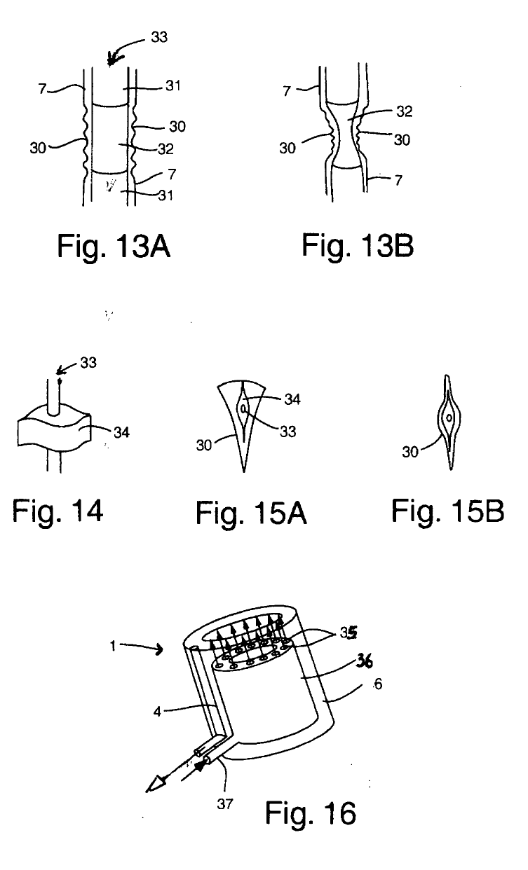

The airway implant device can also be deployed using the following alternative embodiments of the invention as illustrated in FIG. 10, FIG. 11, FIG. 12, FIG. 13, FIG. 14, and FIG. 15. In addition, such illustrations can also describe the shape, structure, and mechanical properties of the tubing and drain that conducts the aspirate away from the trachea.

In one embodiment the drain and associated evacuation tube (exit port) can be positioned through the mouth instead of trachea and so does not damage the vocal folds. Alternatively, the tube can be made of a collapsible material so that the tube can be collapsed until aspirate is removed. The collapsible tube may have a section that is softer and more flexible than the rest so that when the tube is inserted through the vocal folds it collapses, allowing the vocal folds to completely seal at the top of the larynx, therefore allowing normal speech. In this embodiment the collapsing of the tube is passive and does not require an operator.

In another embodiment the device comprises a tube wherein the expansion and contraction of the tube is actively driven by an external pressure/suction unit. The tube element of the device comprises two concentric lumens in at least the region that goes through the vocal folds. The inner lumen is used to remove fluid from the airway implant device. The outer lumen is used only for expansion and collapsing of the tube. Suction is applied to both the inner and outer lumens of the device to collapse the lumens.

In yet another alternative, the tubing is shaped and sized such that it minimizes the air gap between the tubing and the vocal folds when the vocal folds are closed or constricted.

In another alternative embodiment of the invention, the aspirate is actively removed from the collector whereby the individual having aspiration is required to cough or otherwise close and/or constrict the vocal folds. The resulting pressure below the vocal folds forces the aspirate from the collector via the drain to a location away from the trachea. Additional evacuation sites for conducting the aspitrate away from the trachea include, but are not limited to, through the nose, the mouth, the esophagus, the stomach, the intestines, the anus, and the eyeball.

FIG. 10 shows an exemplary embodiment wherein the airway implant device comprises a material that can be operably collapsed and expanded. Such materials are, but not limited to, NITINOL, an elastomeric material such as latex or the like, and a stainless steel stent. FIG. 10A shows the device 1 in a collapsed and non-deployed state positioned in the lumen of the tracheal wall 7. FIG. 10B shows the device in an expanded and deployed state.

FIG. 11 shows three embodiments of an airway implant device comprising a flexible material that can be deployed by operably inflating at least one lumen in the airway implant device. Such flexible materials are described herein. FIG. 11A shows the device comprising a first lumen 28 and a second lumen 29. Prior to use the airway implant device is in a collapsed or uninflated state. In use, at least one lumen of the airway implant device is expanded or inflated using a pressurized gas or the like and the device becomes shaped and sized to be suitably positioned in the trachea to collect aspirate. FIG. 11B shows an alternative embodiment wherein a single lumen is collapsible and inflatable. FIG. 11C shows an alternative embodiment wherein the device comprises two concentric lumens, the first lumen being inflated in use, thereby providing the device with a suitable rigid structure for placement and use in the trachea. In all exemplary embodiments wherein two lumens are herein described, the inflatable lumen can be either the first or the second lumen.

FIG. 12 shows an exemplary embodiment of the device wherein the device comprises a material that can be operable deployed to achieve a preferred shape. In this example, the deployed shape is that of an elipse or torus (see FIG. 12B) and the non-deployed state is that of a circle (see FIG. 12A). Alternatively, the deployed shape can be a circle (see FIG. 12A).

FIG. 13 shows an exemplary embodiment that can be placed in use in the lumen of the vocal folds 30. This is advantageous for the individual having aspiration because the device does not interfere with nor constrain the movement of the vocal folds, hence the individual is able to breath and speak without difficulty. Another advantage is that a device so shaped can be secured by the vocal folds and can also resist movement, such as accidental removal. The device further comprises deploying means 33, such as tubing, that secures the device in place in the trachea, the tubing comprising a non-deformable material 31 and a deformable material 32. FIG. 13 A shows the device in use when the vocal folds are relaxed. FIG. 13B shows the device in use when the vocal folds are constricted, such as occurs during speech, coughing, sneezing, or the like. The deformable material is deformed when the vocal folds constrain upon the external surface.

FIG. 14 shows an exemplary embodiment of the device that can be placed in use through the vocal folds. The device further comprises a molded block 34 that is shaped and adapted for positioning in the lumen of the vocal folds. FIG. 15 shows an illustration from a superior or anterior perspective of the alternative embodiment in use. FIG. 15A shows the device in use when the vocal folds are relaxed. FIG. 15B shows the device in use when the vocal folds are constricted.

The invention also contemplates means for cleaning the airway implant device. Such means can include a tube having a lumen positioned at or adjacent to the collector. A cleansing agent, such as a biological detergent or biological enzyme formulation, is introduced through the lumen of the tube and the cleansing agent flows into the collector and/or channel resulting in a cleaner device. Such an embodiment is advantageous when the device is implanted for an extended period of time.

Additional means for cleaning can include, but are not limited to, mechanical means using external activation and/or airflow activation, a wiping device, a “pipe-cleaner”-like device, an ultrasound device, or the like.

The collector can further comprise a gas-permeable membrane, the gas-permeable membrane covering the lumen of the collector, thereby directing aspirate to the rim of the collector for disposal. Alternatively, a filter and/or screen covers the lumen of the collector, the filter and/or screen having apertures sized to enable only gas to pass through the filter and/or screen.

The surface of the airway implant device can further comprise a coating, the coating comprising drug formulations, drug-eluting formulations, enzyme formulations, and antibiotic formulations. The coating is advantageous for the individual having aspiration and can prevent irreversible and/or injurious attachment of the device to the tracheal mucosal epoithelium as well as preventing attachment of biological compounds produced both from the surrounding tissues or by another agent (for example, a bacterium or fungus). The coating formulation can further comprise a hydrogel. The drug formulations can provide means to deliver asthmas medications, antibiotics, medications used to treat cystic fibrosis, drugs used for chemotherapy.

The airway implant device can also be incorporated into any other clinical or medical device for use in an individual to provide treatment for any condition. Such devices can include, but are not limited to, an endotracheal tube, a “trach” tube, an endoscope, a tracheosope, a catheter, a stent. The implant device can also be used to provide a conduit for draining fluid from another anatomical region, for example, in an individual having hydrocephalus or the like, whereby the fluid is drained from the brain using the device surgically positioned in a suitable anatomical location, such as the neck or thorax.

The invention also contemplates an external indicator or marker that is worn or attached to the individual having the airway implant device so that others can recognize that the individual has the device implanted. This can be advantageous to the individual in an emergency situation. Such an indicator or marker can be, for example, a sharpe or a radiographic marker.

In one embodiment, the flow-diversion device is a hemi-toroidal cup having at least one lateral drainage spout. The shape is essentially that of a donut cut transversely across its circular axis to form a circular cup with a hole in the middle. The drainage spout is positioned at or near the bottom of the cup to allow accumulated fluid to flow out.

The hemi-toroidal cup is placed within the lumen of some part of the oral-tracheal pathway such that the outer surface of the cup is in contact with the interior side of the tube in which it is positioned. Liquids and solids flowing down the tubular portions of the oral-tracheal pathway tend to remain in contact with the walls of the tube within which they are flowing. They do not generally tend to flow through the center lumen of the tube. This is especially so when a small amount of liquid is imbibed, i.e., when the patient is sipping. When gulping, a bolus tends to be formed which then may fill the lumen of the tube, but such gulping behaviour may easily be avoided. Because liquids tend to flow along the surface of the conducting tube, they therefore have a tendency to flow into the hemi-toroidal cup, and out via the drainage spout. The hole in the middle of the hemi-toroidal cup allows gasses to pass through the device allowing normal breathing and speech.

The hemi-toroidal cup may be positioned into the lumen of a tube within the oral-tracheal pathway by any suitable means, for example by means of a tracheal stent disposed about the circumference of the cup. The stent can be placed within the tube and expanded such that the stent and the cup are firmly (but optionally reversibly) positioned within the tube.

### LIST OF REFERENCE NUMERALS

1. Airway Implant Device

2. Channel

3. Lumen

4. Drain

5. Stiffening Element

6. Collector

7. Trachea Wall

8. Adhering Means (Inflatable and Elastomeric)

9. Adhering Means (Foam)

10. Surface of Skin

11. Sutures

12. Apertures

13. Stent Element

14. Laryngoscope

15. Deploying Means

16. Releasable Attaching Means

17. Bronchoscope

18. Eye of Operator

19. Puncturing Means

20. Hook Means

21. Loop

22. First Arm

23. Second Arm

24. Third Arm

25. Baffle

26. Esophagus

27. Laryngopharynx

28. First Lumen

29. Second Lumen

30. Vocal Folds

31. Tubing (Non-deformable)

32. Tubing (Deformable)

33. Deploying Means

34. Molded Block

35. Aperture

36. Reservoir

37. Inlet

38. Fistula

### EXAMPLES

The invention will be more readily understood by reference to the following examples, which are included merely for purposes of illustration of certain aspects and embodiments of the present invention and not as limitations.

### Example I

### Methods of Using Airway Implant Device

1. Percutaneous tracheostomy approach using percutaneous dilational tracheostomy (PDT) method. The advantage of this method of inserting devices into the trachea is that it is cost-effective, safe and easy to perform. The method can be done under local anesthesia. The steps of the method are as follows: The patient is placed on 100% oxygen for about 15 minutes before the procedure.

1) The patient can be placed under sedation (intravenous sedation using fentanyl)

2) Local anesthesia (1% lidocaine with 1:100,000 epinephrine) is applied on the neck under the cricoid cartilage.

3) An incision is made horizontally in the neck.

4) An introducer needle connected to a syringe is advanced, at an angle of 45 degrees to the skin, until air is aspirated from the trachea.

5) The syringe is removed keeping the needle in place. A guidewire is then passed through the needle into the trachea.

6) The introducer needle is then removed and an introducer is passed into the trachea over the guidewire.

7) The guidewire is removed. Now only the introducer is left in the trachea.

8) Tracheal dilators are passed in order, gradually dilating the incision to accommodate the appropriately sized device delivery tube comprising an implantable device.

9) The delivery tube is passed over the introducer into the trachea.

10) The device is deployed and the introducer and the device delivery tube are withdrawn.

The tracheal dilation step can also be done using other translaryngeal methods such as i) Guidewire Dilating Forceps (GWDF) method wherein a set of dilating forceps are advanced over the guidewire and are then opened to dilate a tracheal opening; or ii) Rapitrach technique wherein a Rapitrach dilator is introduced into the trachea over the guidewire and then opened to dilate a tracheal opening.

2. Method using a laryngoscope. In this method, a laryngoscope is used to implant the devices. A laryngoscope comprises a handle, a blade that is inserted through the mouth, a light source located on the distal end of the blade for illuminating the anatomy. The curved blade comprises a groove for inserting devices into the trachea. The blade may be substantially straight, curved or flexible. In an example of a method using a laryngoscope:

1) The mucosa of the oropharynx, and upper airway is anesthetized with local anesthesia (2% lidocaine).

2) The patient's mouth is opened and the blade is advanced into the hypopharynx.

3) The laryngoscope is positioned such that the distal end of the blade is positioned close to the vocal cords.

4) An introducing device comprising an airway implant is then inserted through the groove on the blade such that the airway implant device is positioned in the desired target region in the trachea beyond the vocal cords.

5) Thereafter, the airway implant device is deployed in the trachea and the introducing device and the laryngoscope are removed.

6) If needed, the position of the airway implant device is verified by X-ray radiography.

In one embodiment of the introducing device, the introducing device comprises a removable stylet that provides additional stiffness to the introducing device during the procedure. Alternatively, the stylet may comprise one or more turns or curved regions. In another alternative, the stylet comprises a spiral shaped region.

In another embodiment, the airway implant device is located on the distal region of the laryngoscope and is deployed directly through the laryngoscope.

### Example II

### Testing the Device in an Experimental Model

The purpose of this experiment was to demonstrate proof-of-principle of the device in animal tissue and to measure the maximum rate of fluid flow that can be diverted by the device at different tracheal positions and different suction pressures.

Experimental Setup

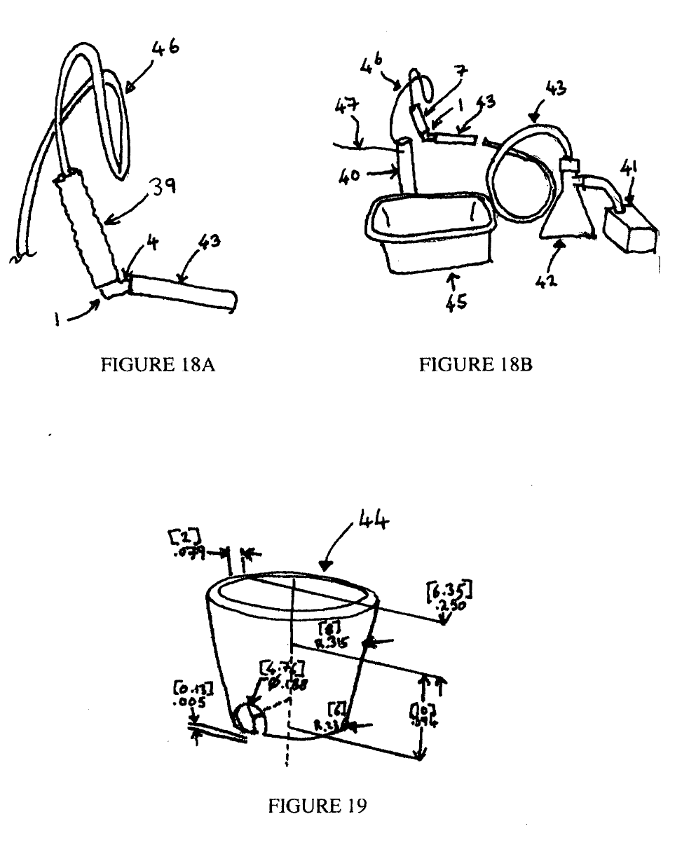

The equipment was setup such that fluid could be introduced into the top of the trachea at a known flow rate and vacuum of known pressure could be applied to the exit port of the device. Photos of the setup are shown in FIG. 18. These photos show the setup when no suction was applied to the device.

Fluid flow was controlled at the sink faucet and measured with the rotameter. The vacuum was provided by the vacuum lines in the lab and regulated with the intermittent suction unit. The continuous suction setting was used on the suction unit during all experiments. The regulated vacuum was connected to the flask to prevent fluid from entering the vacuum lines. A final piece of vacuum tubing connected the flask to the drain port of the device for experiments where suction was used. For experiments that did not use suction, a short piece of vacuum tubing was attached to the drain port and allowed to drain into the basin below the trachea (see FIG. 18).

Equipment

A complete description of the equipment used in the experiment is shown on Table 1.

Airway Implant Device Fabrication

The device used in the experiment was tapered such that it could be cut to the size of the pig trachea. A portion of the mold used to make the device is shown in FIG. 19. The mold was dipped four times in a mixture of four parts red PLASTI-DIP and one part naptha. The outer diameter of the device was 18 mm and fit snugly inside the bottom of the trachea. The outer diameter at the bottom of the device was 12 mm.

Animal Tissue

The pig trachea was prepared by excising removing the tissue around the trachea and cutting it just below the larynx and just above the bronchi. After cleaning the tissue, it had been frozen and thawed twice before the experiment.

Procedure

The trachea was held by a clamp and adjusted to the desired angle relative to vertical. The direction of rotation is similar to placing a patient on his back. The angle and position of the inlet tubing was adjusted to direct the fluid against the back wall of the trachea.

The following procedure was used to measure the vacuum pressure. The vacuum tube connecting the regulator and the flask was removed from the regulator. A finger was placed over the inlet port of the regulator such that there was no flow through the regulator. The vacuum pressure was read once the gauge stabilized. Adjustments of the vacuum pressure were made between measurements while the inlet port was open. Once the desire vacuum pressure was obtained, the vacuum tubing was reconnected.

Water flow was increased using the sink faucet while watching the rotameter reading. The flow rate was adjusted to be at the maximum rate at which there was no visual sign of fluid overflow into the center lumen of the airway implant device. Minimal overflow was observable as a water droplet attached to the bottom of the device grew in size until falling from the device.

Results

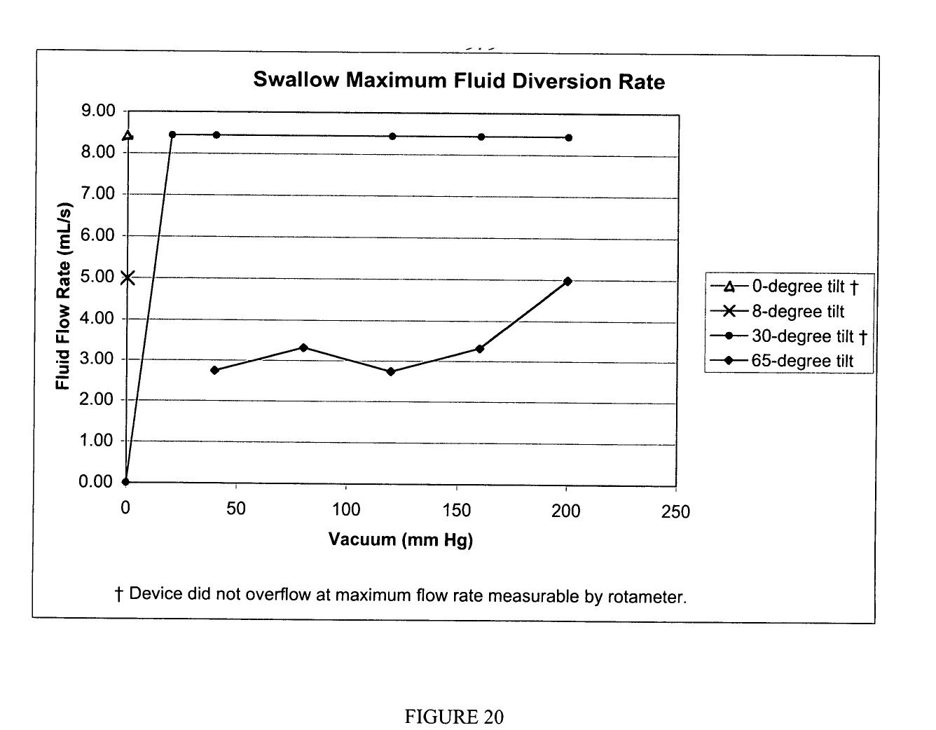

The results are summarized in Table 2 and FIG. 20.

Gravity-driven operation (no suction) was tested with the trachea vertical (0°) and with the trachea rotated such that the Swallow drain port was horizontal (8° trachea angle from vertical). When the trachea was vertical, the maximum flow rate exceeded the flowmeter scale measurement. When the exit port was horizontal, the flow rate was 5 mL/s. When the trachea was angled beyond 8°, gravity-driven operation failed and all fluid overflowed into the central lumen.

Suction-driven operation was tested with the trachea at 30- and 65-degrees from vertical. At 30°, the maximum flow rate exceeded the flowmeter scale measurement at pressures between 20-200 mm Hg. At 65°, the maximum flow rate (5 mL/s) was achieved with the maximum vacuum (200 mm Hg) applied. The maximum flow rate varied between 2.7-3.3 mL/s at pressures between 40-160 mm Hg.

### CONCLUSIONS

The device was shown to operate successfully using water in a pig trachea. Gravity-driven operation was successful when the trachea was vertical. Suction-driven operation was required when the trachea was tilted.

Those skilled in the art will appreciate that various adaptations and modifications of the just-described embodiments can be configured without departing from the scope and spirit of the invention. Other suitable techniques and methods known in the art can be applied in numerous specific modalities by one skilled in the art and in light of the description of the present invention described herein. Therefore, it is to be understood that the invention can be practiced other than as specifically described herein. The above description is intended to be illustrative, and not restrictive. Many other embodiments will be apparent to those of skill in the art upon reviewing the above description. The scope of the invention should, therefore, be determined with reference to the appended claims, along with the full scope of equivalents to which such claims are entitled.

TABLE 1
Equipment

| | | Specifications/Serial |
|---|---|---|
| Description | Make/Model | Number |
| Fluid Tubing | | 5/32″ ID vinyl |
| Vacuum Tubing | | _″ ID, _″ OD |
| | | braided vinyl |
| Rotameter | Omega FL-3839ST | S/N 115478-1 |
| (Flowmeter) | with Tube FT-034- | |
| | 39-ST-VN | |
| Vacuum Regulator | Ohmeda Intermittent | S/N AHBH44455 |
| | Suction Unit | |
| Fluid Reservoir | Erlenmeyer Flask | 1L with side port |
| Airway Implant | — | — |
| Device of the | | |
| Invention | | |

TABLE 2
Maximum flow rates

| ISU | | |
|---|---|---|
| Vacuum | Trachea | |
| Pressure | Angle | Flow Rate |
| (mm Hg) | (°) | (mL/s) |
| 0 | 0 | 8.43† |
| 0 | 8 | 4.98 |
| 200 | 45 | 5.84 |
| 200 | 65 | 4.98 |
| 160 | 65 | 3.32 |
| 120 | 65 | 2.73 |
| 80 | 65 | 3.32 |
| 40 | 65 | 2.73 |
| 200 | 30 | 8.43† |
| 160 | 30 | 8.43† |
| 120 | 30 | 8.43† |
| 40 | 30 | 8.43† |
| 20 | 30 | 8.43† |
| 0 | 30 | 0 |

†Maximum flow rate beyond flowmeter scale. Value shown is maximum flow rate measurable by flowmeter.

We claim:

1. An airway implant device for collecting aspirate the device comprising a collector, the collector further comprising at least one lumen and at least one drain, the device being adapted for placement within the trachea of an individual having aspiration.

2. The airway implant device of claim 1 further comprising at least one stiffening element, wherein the element comprises a material selected from the group consisting of coated TEFLON, high-density polyethylene (HDPE), low-density polyethylene (LDPE), polyamide, nylon, (PTFE), and polyetheretherketone (PEEK).

3. The airway implant device of claim 1 wherein the device comprises a material, the material selected from the group consisting of NITINOL, stainless steel, and titanium.

4. The airway implant device of claim 1 wherein the collector further comprises an elastomeric material, the elastomeric material selected from the group consisting of polyurethane, poly(vinyl)chloride (PVC), silicone rubber, and latex.

5. The airway implant device of claim 1 wherein the collector further comprises a swellable material further comprising a swelling gel.

6. The airway implant device of claim 5 wherein the swelling gel is a super absorbent polymer.

7. The airway implant device of claim 6 wherein the super absorbent polymer is sodium polyacrylate.

8. The airway implant device of claim 1 wherein the collector further comprises securing means, the securing means adapted for securing the collector to the individual's tissue, thereby securing the tracheal implant device within the trachea.

9. The airway implant device of claim 8 wherein the securing means is selected from the group consisting of sutures, clips, hook, staples, stents, and adhesives.

10. The airway implant device of claim 1 wherein the collector further comprises securing means, the securing means adapted for securing the collector to the individual's trachea, thereby securing the airway implant device within the trachea.

11. The airway implant device of claim 10 wherein the securing means is selected from the group consisting of a chemical foam, an inflatable balloon, an inflatable cuff, and a flange.

12. The airway implant device of claim 11 wherein the chemical foam is selected from the group consisting of hydrophilic foams, hydrophobic foams, and biodegradable foams.

13. The airway implant device of claim 11 wherein the chemical foam is selected from the group consisting of polyurethane, polyhydroxyalkanoate (PhA), polyhydroxybutyrate (PhB), PVC, and PTFE.

13. The airway implant device of claim 1 further comprising delivering means, the delivering means having deploying means, the deploying means having releasable attaching means securing the collector.

14. The airway implant device of claim 13 wherein the delivering means is selected from the group consisting of a laryngoscope and a bronchosope.

15. The airway implant device of claim 13 wherein the delivering means comprises a puncturing means, the puncturing means shaped and adapted for puncturing the skin, fascia, cartilage, and tracheal tissue of the individual.

16. The airway implant device of claim 15 wherein the delivering means further comprises a loop.

17. The airway implant device of claim 1 wherein the collector further comprises a first lumen and a second lumen and wherein the collector comprises an elastomeric material.

18. The airway implant device of claim 10 wherein the outermost surface of the collector further comprises securing means comprising a plurality of apertures, the apertures shaped and sized to be in fluid communication with an external vacuum means.

19. The airway implant device of claim 1 further comprising a vacuum pump, wherein the vacuum pump removes the aspirate from the collector using negative pressure.

20. An airway implant device for preventing aspirate from reaching the lungs comprising a collector for collecting aspirate and a drain, wherein the airway implant device is adapted for placement within the airway of a patient at risk of developing aspiration pneumonia.

21. The airway implant device of claim 20, wherein the collector further comprises a lumen.

22. The airway implant device of claim 20 wherein the height of the collector is less than about 2.5 centimeters.

23. An airway implant device comprising a first arm, a second arm, and a third arm, the airway implant device shaped and adapted for placement in the laryngopharynx of an individual having aspiration, and wherein the third arm is further shaped and adapted for diverting aspirate from flowing into the trachea.

24. The airway implant device of claim 23 further comprising a baffle, the baffle shaped and adapted for placement in the laryngopharynx of an individual having aspiration, and wherein the baffle is further shaped and adapted for diverting tracheal aspirate from flowing into the trachea

25. An airway implant device for collecting aspirate the device comprising a collector and at least one drain, the device being adapted for placement within the trachea of an individual having aspiration.

26. A method of using the airway implant device of claim 1 to treat an individual having aspiration and that results in the patient having an improved ability to talk and to breathe, the method comprising the steps of:
(i) providing an individual having aspiration,
(ii) providing the airway implant device of claim 1,
(iii) attaching the airway implant device to attaching means,
(iv) further attaching the attaching means to deploying means,
(v) implanting the airway implant device in the trachea of the individual using deploying means,
(vi) releasing the airway implant device from the attaching means, and
(vii) securing the airway implant device to the wall of the trachea using securing means, thereby treating the individual having aspiration and results in the patient having an improved ability to talk and to breathe.
